(19) United States
(12) Patent Application Publication
(10) Pub. No.: US 2009/0117161 A1
(43) Pub. Date: May 7, 2009

(54) SEMICONDUCTOR-BASED CORE-SHELL PARTICLES FOR BLOCKING ELECTROMAGNETIC RADIATION

(76) Inventors: KONSTANTINOS P. GIAPIS, Pasadena, CA (US); Nicholas A. Brunelli, Pasadena, CA (US); Maria Lambros, Upland, CA (US)

Correspondence Address: CHRISTIE, PARKER & HALE, LLP, PO BOX 7068, PASADENA, CA, 91109-7068, US

(21) Appl. No.: 12/134,185

(22) Filed: Jun. 5, 2008

Related U.S. Application Data
(60) Provisional application No. 60/933,321, filed on Jun. 5, 2007.

(51) Int. Cl. A61K 8/25; A61K 8/02; B82B 1/00; B82B 3/00

(52) U.S. Cl. 424/401; 424/59; 977/926; 977/840; 977/700

(57) ABSTRACT

A plurality of particles for blocking electromagnetic radiation wherein each particle includes at least one semiconductor core encased within a shell, the semiconductor cores being of substantially uniform diameter, which diameter is selected according to the quantum size effect such that radiation incident on the particles is absorbed below a preselected radiation wavelength. In particular embodiments, the diameter may not vary between cores by more than a preselected percentage, and the diameter may fall within the range of approximately one to approximately five nanometers. Each particle may have a single semiconductor core surrounded by a single shell or a plurality of semiconductor particles surrounded by a single shell. In other embodiments, the particles may be created by forming at least one semiconductor core in a first reaction zone and forming a shell encapsulating the at least one semiconductor core in a second reaction zone. A plurality of semiconductor cores may optionally be agglomerated together before the shell is formed.

### CROSS-REFERENCE TO RELATED APPLICATION(S)

This application claims priority to U.S. Provisional Application Ser. No. 60/933,321, filed on Jun. 5, 2007, the entire content of which is incorporated herein by reference.

### FIELD OF THE INVENTION

The invention is directed to the use of semiconductor-based core-shell nanoparticles for blocking selected wavelengths of electromagnetic radiation.

### BACKGROUND OF THE INVENTION

Sunscreens have long been used to block the UV rays that cause skin damage. The shorter UVB wavelengths are the principle cause of sunburn, though UVA wavelengths can penetrate and harm deeper layers of the skin. Because both UVA and UVB wavelengths cause skin damage, an effective sunscreen should block both. Traditionally, chemical sunscreens and physical sunblocks are effective against either UVA or UVB, so a combination of different ingredients is required for protection against the full UV spectrum.

Chemical sunscreens and physical sunblocks each have disadvantages. Chemical sunscreens are generally large organic molecules that absorb certain wavelengths of UV. Although they can be formulated to cover specific bands of the UV spectrum, they can trigger skin sensitivity or allergic reactions, or affect hormonal activity. Some chemical sunscreens are affected by exposure to the sun and degrade after some time, so there is a need to add chemical stabilizers to the sunscreen formulation. In the United States, chemical sunscreens are regarded as drugs, so development of new sunscreens is limited by the drug regulation process.

Physical sunblocks are typically micronized particles of zinc oxide or titanium dioxide about 500 to 2000 nm in size. Titanium dioxide protects only against radiation wavelengths shorter than 360 nm, while zinc oxide protects against radiation wavelengths shorter than 380 nm. Since the UVA band ranges from 315-400 nm, and some suggest total protection up to 420 nm, the current physical sunblocks are insufficient to fully protect from the total harmful spectrum of sunlight. Although these particles are thought to be less irritating than chemical sunscreens, they are more difficult to spread evenly on the skin and tend to leave the skin chalky and white, both cosmetically unacceptable consequences. Newer formulations use nanoparticles, solving the cosmetic issues but raising possible health concerns. Particles under 40 nm are suspected to be able to penetrate the skin, where they can potentially cause subcutaneous damage. Both zinc oxide and titanium dioxide are photoactive, meaning they catalyze chemical reactions when exposed to light. Because the high surface area of such nanoparticles provides many catalytic sites, there is the possibility that the nanoparticles themselves can cause skin damage, including damage to cellular DNA. Furthermore, there is evidence that titanium dioxide nanoparticles are taken up by the roots of trees and interrupt plant growth. Because sunscreen use is widespread and ever increasing, environmental considerations are important in sunscreen development.

### SUMMARY OF THE INVENTION

In one embodiment of the present invention, a semiconductor nanoparticle core that prevents transmission of light below a specified wavelength is encapsulated by a biologically inert shell. The core-shell nanoparticle is small enough to be spread easily on the skin without creating a chalky white film, yet large enough to prevent diffusion through the outer skin layer or penetration into cells. In another embodiment of the invention, a single shell encapsulates several semiconductor nanoparticle cores of the same size.

In another embodiment of the invention, the above-described particles may be suspended in a cream or lotion and applied to skin for protection against UV wavelengths from sunlight.

Still another embodiment of the invention includes the method for producing the above-described particles.

Thus, the invention includes a plurality of particles for blocking electromagnetic radiation, each particle having at least one semiconductor core encased within a shell, the semiconductor cores being of substantially uniform diameter, such diameter selected according to the quantum size effect such that radiation incident on the particles is absorbed below a preselected radiation wavelength. In one embodiment, the diameter of the semiconductor cores does not vary between semiconductor cores by more than a preselected percentage. In another embodiment, the diameter may fall within the range of approximately one to approximately five nanometers, possibly approximately four or approximately three nanometers. In further embodiments, each particle may include a single semiconductor core surrounded by a single shell, or a plurality of semiconductor cores surrounded by a single shell. The core may also be include silicon and the shell may include a compound containing silicon.

The invention may also include a method of forming particles having at least one semiconductor core encased within a shell, the method comprising; forming the at least one semiconductor core in a first reaction zone; and forming a shell encapsulating the at least one semiconductor core in a second reaction zone. In one embodiment, the semiconductor cores can be processed by agglomerating a plurality of semiconductor cores together prior to formation of the encapsulating shell.

### BRIEF DESCRIPTION OF THE DRAWINGS

The above and other features and advantages of the present invention will be better understood by reference to the following detailed description when considered in conjunction with the accompanying drawings in which:

FIG. 1 is a schematic of a core-shell particle, where the core has a diameter d and the shell creates a particle with diameter D;

FIG. 2 is a schematic of a multicore-shell particle, where each core particle has a diameter d and the shell encapsulates more than one core to create a particle with diameter D;

FIG. 3 is an absorbance spectrum of an ideal sunscreen containing particles of uniform size showing a sharp absorption edge at 400 nm;

FIG. 4 is an absorbance spectrum of a sunscreen containing nanoparticles of varying size showing a gradual decline in absorbance at increasing radiation wavelengths;

FIG. 5 is a plot showing absorbance spectra of Si/SiOₓ core-shell nanoparticles grown from silane/Ar in an inductively-coupled plasma and deposited for 30, 60, and 120 minutes on a quartz substrate, where the cores are of approximately the same size and the spectra show fairly sharp declines in absorbance around 400 nm;

FIG. 6 is a schematic of a two-zone reactor for producing core-shell nanoparticles;

FIG. 7 is a schematic of a four-zone reactor for producing multicore-shell nanoparticles.

### DETAILED DESCRIPTION OF THE INVENTION

The present invention is directed to the use of core-shell nanoparticles having semiconductor cores of substantially uniform diameter to block selected wavelengths of electromagnetic radiation. The size of the semiconductor cores determines which radiation wavelengths are blocked, while the shell thickness and composition establish the size and surface characteristics of the overall particle. The independent nature of the semiconductor core and the shell allows a particle to be designed to meet specific needs.

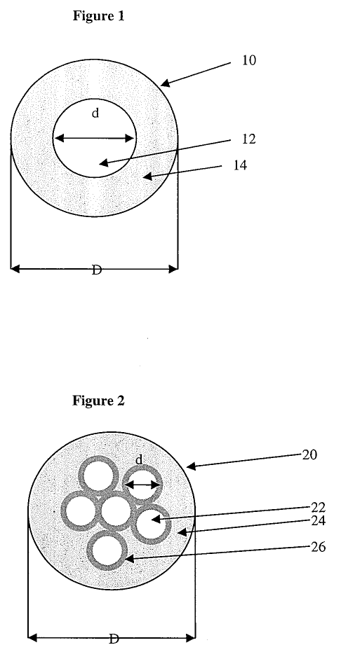

In one embodiment, a core-shell particle 10 includes a semiconductor core 12 encapsulated by a shell 14, as shown in FIG. 1. In another embodiment, a multicore-shell particle 20 includes a plurality of semiconductor core particles 22 encapsulated by a shell 24, as shown in FIG. 2. The semiconductor core particles 12 and 22 each have a diameter substantially equal to d, and are composed of silicon, germanium or any other material or combination of materials suitable for blocking transmission of electromagnetic radiation. Suitable materials for the semiconductor core 12 or 22 include materials that exhibit the quantum size effect, can be made into core particles of the appropriate size, and block the transmission of radiation of the desired wavelengths. One method of blocking transmission of electromagnetic radiation is by absorption. A suitable material for the semiconductor core 12 or 22 may be tuned to efficiently absorb light below a specified radiation wavelength by adjusting core size or by doping, which is the addition of other materials. The shells 14 and 24 each have an outer diameter D, and are composed of silicon dioxide, silicon nitride, aluminum oxide, titanium oxide or any other suitable material or combination of materials. Suitable materials for the shell 14 or 24 include materials that allow at least partial transmission of radiation wavelengths that are absorbed by the cores 12 or 22; are inert to air, skin and oils; prevent or reduce oxygen diffusion into the semiconductor core; or impart other desirable characteristics in the core-shell particle 10 or 20. In the multicore-shell particle 20, each semiconductor core 22 has a protective layer 26 to prevent the semiconductor cores 22 from fusing together to create larger particles. This change in particle size would affect the absorbance spectrum of the particle. The protective layer 26 may be the same material as the exterior shell 24 or other suitable material. Suitable materials for the protective layer 26 include materials that prevent the bare surfaces of the semiconductor cores 22 from fusing and allow for agglomeration of the semiconductor cores into clusters.

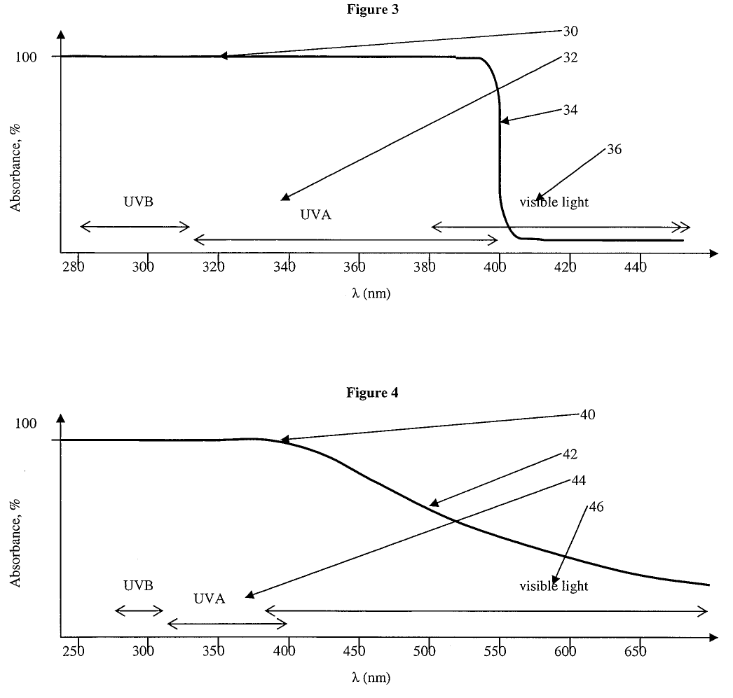

The absorption edge for a single particle is the wavelength of electromagnetic radiation at which absorption by the particle sharply increases. Typically a material absorbs all wavelengths of radiation shorter than the critical wavelength defined by the absorption edge. An ideal sunscreen would be one that blocks all harmful UV wavelengths but does not block any visible light. If the sunscreen blocks visible light, the color of the skin can change according to the wavelengths of visible light affected by the sunscreen. Therefore, the absorbance spectrum 30 of an ideal sunscreen would resemble a step function, as represented in FIG. 3. The absorbance spectrum 30 shows complete absorbance of radiation wavelengths corresponding to UVB and UVA regions 32. Absorbance edge 34 shows a sharp decline in absorbance around 400 nm, the upper bound of the UVA region. The absorbance spectrum 30 shows no absorbance in the visible light region 36.

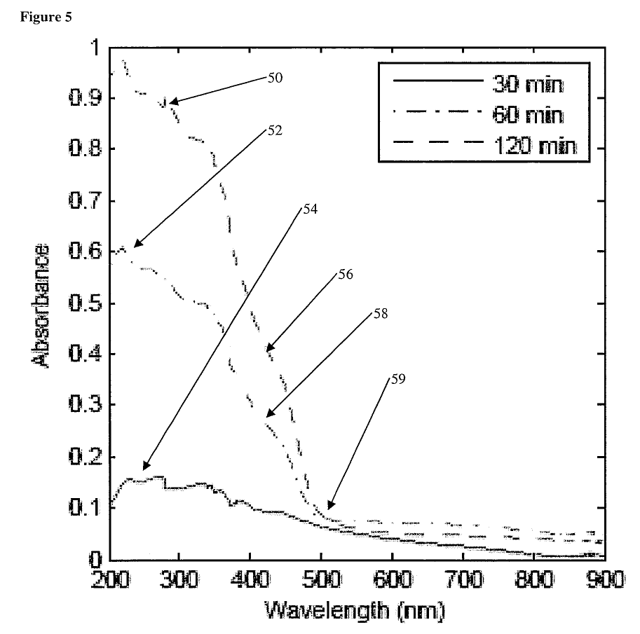

As set forth below, at small particle sizes, the absorption edge is a function of semiconductor core particle size due to the quantum size effect. If the bulk of the core particles was of substantially uniform size, small amounts of differently-sized particles would not significantly change the absorbance spectrum. However, a sunscreen containing a large distribution of semiconductor core particle sizes has a corresponding distribution of individual absorption edges. This results in an overall spectrum 40 with a gradual decrease in absorbance 42 as radiation wavelength increases, as shown in FIG. 4. In order to get full protection from UV wavelengths 44 in a sunscreen containing a large distribution of semiconductor core particle sizes, the sunscreen would have to be designed to block out some visible light 46. Such a sunscreen would be cosmetically unacceptable because it would change the color of the skin. For example, if the sunblock absorbed violet and blue light, the skin would appear have a yellowish tint because the absorbed light wavelengths would not be available for reflection by the skin. If the sunblock absorbed all visible light, the skin would appear black. To address this issue, one embodiment of the present invention is a sunscreen comprised of semiconductor core particles of substantially uniform size. When in a dispersed phase, these similarly-sized particles can be referred to as “monodisperse.” This monodisperse product retains the desired sharp absorption edge of a single semiconductor core particle, so only harmful UV rays are blocked and skin color remains unaffected. FIG. 5 shows the absorbance spectra of Si/SiOₓ core-shell nanoparticles grown from a silane/Ar reaction in an inductively-coupled plasma. The shell oxide was formed by spontaneous oxidation in ambient conditions. Spectra 50, 52 and 54 show absorbance of radiation by particles deposited on a quartz substrate for 120, 60 and 30 minutes, respectively. The spectra 50 and 52 show fairly sharp declines in absorbance 56 and 58, respectively, around 400 nm. The spectra 50, 52 and 54 all show very little absorption in the visible light region 59.

By selecting a semiconductor core material that both exhibits a quantum size effect and can be made into nanoparticles of uniform size, the core-shell product may have a tunable absorbance spectrum with a sharp cutoff. This is a desirable property for a sunscreen because it completely blocks harmful UV rays without affecting skin color. One advantage of this method over current sunscreen formulations is that in one embodiment only one active ingredient is required to block the entire spectrum of harmful rays.

In an embodiment of the invention, the semiconductor core 12 or 22 of the particle 10 or 20 is made of silicon. Silicon is the second most abundant element in the Earth's crust. Due to the widespread use of silicon in the electronics industry, the requisite precursors and techniques for making nanoparticles are readily available. Silicon is a semiconductor, and like all semiconductors, there is a quantum size effect at small particle sizes wherein a deviation from bulk properties occurs. The energy band gap increases as particle size decreases, and there is a corresponding decrease in the radiation wavelengths absorbed. This means that the absorbance spectrum for a silicon nanoparticle may be tuned by adjusting the size of the particle itself. The size of the particle can be changed in several ways. One method is to increase residence time in the reactor to allow the particle to grow larger. Another is to oxidize the surface of the particle to reduce the effective size of the core material. Still another method of changing particle size is to allow small particles with bare surfaces to cluster together and fuse into a larger particle. A further benefit of using silicon is that any particles released into the environment naturally degrade into silicon dioxide, or sand. The timescale for degradation depends on the thickness and composition of the shell, but oxygen diffusion through any shell will eventually occur.

In one embodiment using silicon, the size of the semiconductor core 12 or 22 required to put the absorption edge 34 around the UV-visible light spectrum is a dimension chosen from the approximate range 1-5 nm. The preferred semiconductor core size range is between 2-4 mm or more specifically, 2.5-3.5 nm. The semiconductor core diameter may be chosen from a range, but all of the semiconductor cores must be substantially uniform in diameter. Substantially uniform means that the majority of particles will deviate from the mean diameter by no more than a specified percentage. That percentage ranges from 5-25%. Particles of this small length scale may be able to penetrate the skin to cause subcutaneous damage. Although silicon itself is inert, the effects of any particles of such small scale are not yet well understood. Further, silicon is readily oxidized to silicon dioxide in the presence of oxygen at ambient conditions, so it is impossible to expose skin to bare silicon. The formation of an oxide layer also decreases the effective size of the semiconductor core, thus affecting the absorption edge. To protect the integrity of the semiconductor core 12 or 22 and to increase the size of the overall core-shell particle 10 or 20 to prevent subcutaneous penetration, a shell 14 or 24 is grown to encapsulate the semiconductor core 12 or 22.

In one embodiment, the shell 14 or 24 can be a silicon oxide. Silicon oxides are biologically inert and do not trigger skin sensitivity or allergies. Also known as silica, it is the principal component of glass and sand, and is even used in food applications as a flow agent for powders. Silicon oxides form easily on silicon surfaces. The prevalence of silicon oxide use in the electronics industry has led to the vast availability of both raw materials and techniques for its precise manufacture. In one embodiment, the silicon dioxide shell 14, 24 or 26 may be grown as large as desired and may be designed to become more or less hydrophilic. It is naturally hydrophilic, which makes it more difficult to penetrate the skin. The surface of the shell 14 or 24 may also be altered by chemical or physical means to provide other desired characteristics. It is therefore possible to make a core-shell particle 10 or 20 with a semiconductor core 12 or 22 of particular size for selective radiation absorption, yet independently change other characteristics of the core-shell particle 10 or 20 by tuning the size and composition of the shell 14 or 24.

The shell 14 or 24 can be of any thickness. In one embodiment, the minimum thickness of a silicon dioxide shell 14 or 24 on a silicon core 12 or 22 is about 5 nm. This is the size at which oxygen diffusion through the silicon dioxide shell 14 or 24 is slow enough that the silicon core 12 or 22 is effectively protected from further oxidation and will therefore remain constant in size over the lifetime of the product. In another embodiment, the shell 14 or 24 is sized such that the core-shell particle 10 or 20 is too large for subcutaneous absorption. The preferred size of core-shell particle 10 or 20 would be between about 40 nm, the size at which particles can no longer be subcutaneously absorbed, and 100 nm, the size at which the particle begins to scatter visible light and create a white or chalky film on the skin. In one embodiment, the core-shell particle 10 or multicore-shell particle 20 may be of a size and surface characteristic such that the core-shell particles 10 or multicore-shell particles 20 spontaneously aggregate in suspension to create core-shell or multicore-shell particle bunches that are large enough to prevent subcutaneous penetration. In such cases, the individual core-shell particles 10 or multicore-shell particles 20 may be as small as 10 nanometers.

In one embodiment, the multicore-shell particle 20 may be composed of several semiconductor cores 22 encased within a single shell 24. This increases the absorption capacity of each multicore-shell particle 20 since there are more radiation-absorbing semiconductor cores 22 per unit volume. However, the effective size of each semiconductor core 22 may remain the same, so the sharp absorption edge 34 is unaffected. One method of making the multicore-shell particles 20 is to make the semiconductor cores 22, grow a protective layer 26 on each, aggregate the semiconductor cores 22 into small clusters, then finally grow another shell 24 to encapsulate the entire cluster. A protective layer 26 is grown on each semiconductor core 22 to prevent them from fusing to form larger particles, which would affect the absorption characteristics. The size of the overall multicore-shell particle 20, established by the size of the cluster and the thickness of the final shell 24, determines whether it can be subcutaneously absorbed or form a chalky residue on the skin. The number of semiconductor cores 22 in the multicore-shell particle 20 is limited by the number of semiconductors 22 that can physically fit within the specified size of the multicore-shell particle 20. There may be as few as two semiconductor cores 22 within the shell 24 to create a multicore-shell particle 20. The final shell 24 may be adjusted to impart different surface characteristics in the overall multicore-shell particle 20.

One embodiment of the invention is a process for creating silicon core particles 12 or 22 with a narrow size distribution. One method for producing silicon core particles 12 or 22 of substantially uniform size is in a plasma reactor using silane gas (SiH₄) in an aerosol reaction. In one embodiment of this invention, the reactor consists of a quartz tube 1″ in diameter and 6″ in length, wrapped with an inductive coil and mechanically pumped down to a pressure of 100 mTorr. The coil is used to deliver 100 W of power through a matched network to an inductively coupled plasma formed inside the tube. 20 ppm of silane in argon serves as the precursor to particle formation and is passed through the tube. Alternate reaction parameters include increasing the quartz tube up to 4″ in diameter, increasing the operating pressure up to 10 Torr, increasing power inputs ranging up to 2000 W, and increasing precursor concentration up to 4% silane in argon.

There are several ways to grow an encapsulating layer 14, 24 or 26, such as an oxide layer on a semiconductor material. The choice of method may affect the properties of the oxide. One method for silicon is natural oxidation. Silicon is highly reactive with oxygen at ambient conditions, and an oxide layer up to about 5 nm thick will spontaneously form on a silicon surface. The rate of oxidation increases exponentially with temperature according to kinetic rate laws and. increasing oxygen concentration also increases the oxidation rate. Oxygen diffuses through each subsequently formed silicon dioxide layer to react with the silicon beneath. However, this native oxide layer 14 or 26 is formed out of the silicon base itself, and therefore reduces the size of the silicon core 12 or 22. One could design this into the process by starting with a semiconductor core 12 or 22 slightly larger than the size required for the desired absorption edge, such that subsequent conversion of semiconductor into an oxide would reduce the semiconductor core 12 or 22 to the intended size. But a remaining disadvantage of this process is that the native oxide layer 14 or 26 cannot be greater than about 5 nm thick. Diffusion of oxygen through an oxide layer of that thickness is slow enough that it would be impractical to grow a thicker layer by this method.

In order to grow a silicon oxide layer thicker than 5 nm, one must employ a different technique. Shell 14, 24 or 26 overgrowth may be accomplished in a reactor using either tetraethyl orthosilicate (TEOS, Si(OCH₂CH₃)₄) or a mixture of silane and oxygen (SiH₄+O₂). The reactor for either technique may be a quartz tube in a furnace or a plasma gas reactor. Jet injection/turbulent mixing of the precursor, either TEOS or oxygen, will lead to more uniform oxide growth. Silicon oxidation may also be carried out with molecular oxygen (O₂). The shell 14, 24 or 26 produced by this method may be up to 20 nm thick.

Agglomeration of particles will occur spontaneously because of the small scale. To control to size of the clusters formed, it is crucial to control the residence time in the agglomeration zone of the reactor. Residence time will depend upon particle concentration, particle size, particle size distribution, gas pressure and gas temperature. Temperature and particle concentration may also be used to control the size of the clusters formed. Increasing temperature, particle concentration and particle size distribution leads to faster agglomeration while decreasing gas pressure increases agglomeration for the particle size of interest. As particles emerging from a plasma are usually charged, a corona discharge or charge neutralization source may be necessary in this zone to enable particle agglomeration.

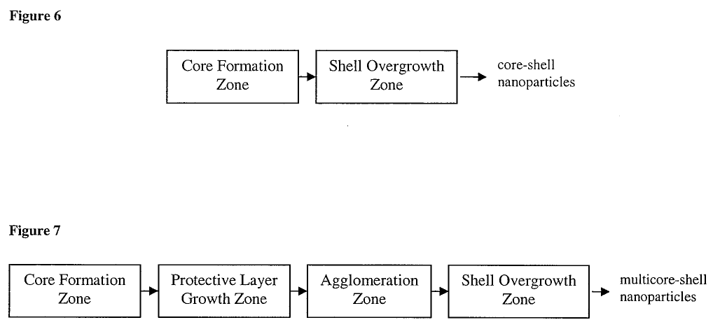

In one embodiment of the invention as shown in FIG. 6, a reactor used to produce core-shell particles 10 includes a) a short reaction zone for forming monodisperse semiconductor cores 12 and b) a shell 14 overgrowth zone to accomplish the desired final size. The zones may be either separate regions of a single reactor or separated into distinct reactors. Those with ordinary skill in the field will understand that permutations of this scheme are possible and that some characterization, adjustments and calibration are required to get the desired result.

In one embodiment of the invention as shown in FIG. 7, a reactor used to produce multicore-shell particles 20 includes a) a short reaction zone for forming monodisperse semiconductor cores 22, b) a zone for growing a protective layer 26 around each semiconductor core, c) an adjustable afterglow zone to permit controlled agglomeration of particles into clusters and d) a shell 24 overgrowth zone to accomplish the desired final size. The zones may be either separate regions of a single reactor or separated into distinct reactors. Those with ordinary skill in the field will understand that permutations of this scheme are possible and that some characterization, adjustments and calibration are required to get the desired result.

What is claimed is:

1. A plurality of particles for blocking electromagnetic radiation, each particle comprising at least one semiconductor core encased within a shell, the semiconductor cores being of a substantially uniform diameter, said diameter selected according to the quantum size effect such that radiation incident on the particles is absorbed below a preselected radiation wavelength.

2. The plurality of particles of claim 1 wherein said diameter does not vary between semiconductor cores by more than approximately 25 percent.

3. The plurality of particles of claim 2 wherein the said diameter falls within the range of approximately one to approximately five nanometers.

4. The plurality of particles of claim 3 wherein the said diameter is approximately four nanometers.

5. The plurality of particles of claim 3 wherein the said diameter is approximately three nanometers.

6. The plurality of particles of claim 1 wherein each particle comprises a single semiconductor core surrounded by a single shell.

7. The plurality of particles of claim 1 wherein each particle comprises a plurality of semiconductor cores surrounded by a single shell.

8. The plurality of particles of claim 1 wherein the shell allows at least partial transmission of radiation wavelengths below a preselected radiation wavelength.

9. The plurality of particles of claim 1 wherein the semiconductor core comprises silicon and the shell comprises a compound containing silicon.

10. The plurality of particles of claim 9 wherein the shell comprises a silicon oxide.

11. The plurality of particles of claim 9 wherein the shell comprises a silicon nitride.

12. The plurality of particles of claim 1 wherein the diameter of the particles is at least approximately 40 nanometers.

13. The plurality of particles of claim 12 wherein the diameter of the particles is no more than approximately 100 nanometers.

14. The plurality of particles of claim 1 wherein the diameter of the particles is no more than approximately 10 nanometers.

15. A method of forming a plurality of particles having at least one semiconductor core encased within a shell, the method comprising;
forming the at least one semiconductor core in a first reaction zone; and
forming a shell encapsulating the at least one semiconductor core in a second reaction zone.

16. The method of claim 15 wherein the at least one semiconductor core is formed by an aerosol synthesis reaction using a gas plasma.

17. The method of claim 16 wherein the gas plasma comprises silane.

18. The method of claim 17 wherein the shell is formed using a gas plasma.

19. A method of forming a plurality of particles each having at least one semiconductor core encased within a shell, the method comprising;
forming semiconductor cores in a first reaction zone;
agglomerating a plurality of semiconductor cores together; and
forming a shell encapsulating said plurality of semiconductor cores in a second reaction zone.

20. The method of claim 19 wherein a protective layer is grown on the semiconductor cores prior to agglomeration of a plurality of said semiconductor cores.
